(19) United States
(12) Patent Application Publication
(10) Pub. No.: US 2006/0173443 A1
(43) Pub. Date: Aug. 3, 2006

(54) Delivery system for therapy comprising hollow seed, preferably polymeric

(76) Inventors: Anatoly Dritschilo, Bethesda, MD (US); Mira Jung, Gaithersburg, MD (US); Manny R. Subramanian, Frederick, MD (US)

Correspondence Address: Ning Yang, 7643 Fullerton Road, Springfield, VA, 22153, US

(21) Appl. No.: 11/368,443

(22) Filed: Mar. 7, 2006

Related U.S. Application Data
(62) Division of application No. 09/382,794, filed on Aug. 25, 1999.

(51) Int. Cl. A61K 9/22

(52) U.S. Cl. 604/890.1

(57) ABSTRACT

Hollow metal and polymeric containers (or seeds) are provided having a therapeutic agent encapsulated therein, e.g., a nucleic acid or cytokine, that diffuses out of the seeds via one or more holes disposed therein and is thereby delivered to target sites, e.g., tumor cells. These hollow seeds can be precisely delivered to garget cites, e.g., within a tumor, preferably by use of stereotactic guidance, ultrasound, CT or MRI.

### CROSS-REFERENCE TO RELATED APPLICATIONS

The present application is a division of application Ser. No. 09/382,794, filed Aug. 25, 1999, currently pending. The present application is related to copending application, titled DELIVERY SYSTEM FOR THERAPY COMPRISING HOLLOW SEEDS, AND THE METHOD OF USE THEREOF, filed on the same date as the present invention by inventor Anatoly DRITSCHILO, Mira JUNG and Manny R. SUBRAMANIAN (attorney docket number 2005-01Div-m). All of these applications are incorporated herein by reference.

### FIELD OF THE INVENTION

The invention relates to a novel delivery system for nucleic acid sequences, e.g., plasmids, antisense or sense oligonucleotides, viral vectors, et seq., that comprises hollow seeds, preferably metal seeds, having encapsulated therein a nucleic acid sequence or other non-radionuclide active agent, preferably a cytokine, toxin or a combination thereof, that elicits a therapeutic effect at a target site, e.g., tumor, and optionally another therapeutic agent, e.g., a radionuclide or other cytotoxic agent. In a particularly preferred embodiment, the nucleic acid sequence will encode a radiation sensitizing gene. The invention further relates to the use of such hollow seed, preferably metal, delivery system as a therapeutic, in particular for the treatment of tumors.

### BACKGROUND OF THE INVENTION

A significant problem of current cancer therapies is providing methods that facilitate selective killing of cancer cells without eliciting substantial non-specific cytotoxicity, i.e., killing of normal (e.g., non-cancerous) cells. Toward that end, various approaches have been developed including chemotherapy, radiotherapy, immunotherapy, and gene therapy. For example, immunotoxins have been developed that target cytotoxic agents to a desired site, e.g., an antigen expressed on a tumor cell. Also, the administration of nucleic acid sequences that target specific genes expressed by tumor cells is known.

Of the various approaches, including chemotherapy, immunotherapy and gene therapy, the latter appears to offer this potential, but practical limitations of gene delivery have presented obstacles that prevent easy implementation. Systemic administration of genetically engineered vectors offers treatment for primary and metastatic diseases. However, the physiology of tumors presents many of the same hurdles faced by chemotherapeutic approaches, particularly heterogeneously perfused tumors with resultant under dosed regions.

The introduction of DNA vectors capable of expression in human cells forms the basic premise of gene therapy. The complexity of vectors that are capable of carrying DNA into cells ranges from plasmids, independent self-replicating circular DNA molecules, to adeno and herpes viruses. Typically, genetic engineering is used to modify the viral genes to make viruses incapable of replication.

Various vectors have been developed to deliver genes to cancer cells for expression of cytotoxic or radiation sensitizing agents. The delivery of these vectors has frequently employed direct injection of virus containing solutions into tumors. At present, this is a slow and poorly controlled process, which leads to a non-uniform deposition of the reagents within the tumors. This intratumoral delivery of genes may involve injection into single or multiple locations throughout the tumor volume. The delivery of genes or cytokines into a tumor offers a particularly attractive option.

Radiation sensitization of tumors, particularly large tumors, has been a long term goal, but effectiveness has been limited in part by tumor physiology. For example, U.S. Pat. No. 4,891,165 describes the encapsulation of radioactive materials in two interlocking metal sleeves made of metallic substances such as titanium, gold, platinum, stainless steel, tantalum, nickel alloy or copper or aluminum alloys. U.S. Pat. No. 4,994,013 discloses a radioactive seed pellet comprising a metallic rod coated with binder material, which is radioactive absorbing. U.S. Pat. No. 5,713,828 describes a seed-shaped substrate comprising a hollow outer metal or synthetic tube coated with radioactive material for use at tumor sites. The hollow tube has openings or perforations as well as open ends in order to pass surgical equipment such as needles there through. All of the above “seeds” are implanted at the affected site then irradiated.

Other methods of delivering either drugs or genetic material to a tumor site for radiation sensitization include those disclosed by U.S. Pat. No. 5,756,122, disclosing liposomally encapsulated nucleic acids. High molecular weight polynucleotides such as antisense DNA are encapsulated and delivered to the tumor site. U.S. Pat. No. 4,674,480 also discloses an encapsulated drug or nucleic acid delivery to a tumor site. Encapsulation is done within protein, fat, cell tissue or a polymer. The desired encapsulated drug or nucleic acid is released by irradiation of thermal decomposition.

The mechanics of interstitial delivery of seeds and encapsulated material as described above have been previously developed for use in radiation therapy for placement of brachytherapy sources. For example, cancers of the prostate, head and neck, breast, pancreas, and sarcomas are routinely treated by placement of encapsulated radioactive pellets uniformly throughout tumor volumes. Recently, ultrasound guided, trans-perineal radioactive seed placement for the treatment of prostate cancer and stereotactically-guided radioactive seed placement for brachytherapy for the treatment of glioblastomas has been developed. Hohn H. H., Juul N., Pedersen J. F., Hansen H., Stroyer I., Transperineal ¹²⁵iodine seed implantation in prostate cancer guided by transrectal ultrasonography, J. Urol., 130:283-286, 1983; Blasko J. C., Radge H., Schmacher D., Transperineal percutaneous Iodine-125 implantation for prostatic carcinoma using transrectal ultrasound and template guidance. Endocurie/hypothermia Oncol., 3:131-139, 1987; Hilaris B. S., Evolution and general principles of high dose rate brachytherapy, In Nag S (ed): High dose rate brachytherapy: A textbook, Futura Publishing Company Inc., Armork, N.Y. 1994. One company in particular, Best Industries, Inc., has been a leader in the area of design, development and manufacture of radioactive isotopes containing metal seeds.

However, to the best of the inventors' knowledge, the use of such hollow seed delivery system for the delivery of nucleic acid sequences to a target site, e.g., a tumor cell has never been suggested. Rather, previous methods for effecting gene delivery have included, by way of example, liposomal delivery systems, the introduction of cells that express desired nucleic acid sequences, and the direct injection of naked DNA, e.g., viruses or antisense oligonucleotides at a target site, e.g., a tumor. As noted above, such delivery methods have typically been ineffective because they are slow and not readily controlled. This is undesirable, as the therapeutic nucleic acid sequence typically does not reach all the desired sites, e.g., cells in a tumor.

### OBJECTS OF THE INVENTION

It is a primary object of the invention to obviate the problems of conventional methods and materials for in vivo delivery of nucleic acid sequences to target sites, e.g., a tumor.

It is a more specific object of the invention to provide a novel system for in vivo delivery of nucleic acid sequences, e.g., viruses, that comprises small hollow seeds, preferably metal or polymeric, having encapsulated therein at least one nucleic acid sequence, e.g., a virus, that elicits a therapeutic effect, and optionally another therapeutic agent, such as a radionuclide.

The invention provides novel methods for effecting gene therapy whereby a desired nucleic acid sequence, e.g., contained in a virus, is delivered to a target site by encapsulating same in a small hollow seed, preferably made of a metal or polymeric material, that may be precisely inserted into the target site (e.g., tumor) by methods such as the use of implantation gun, catheter, syringe, and the like, and further including stereotaxy, ultrasound, CT and MRI guidance thereby confirming efficient, uniform, interstitial distribution of hollow seeds and delivery of nucleic acid sequences contained therein.

The invention also provides a novel method for treating tumors by combined administration of a radiation sensitizing gene and ionizing radiation, by the use of small hollow seeds, preferably made of metal or polymeric material, that provide for the delivery of encapsulated radiation sensitizing genes and ionizing radiation, wherein the radiation sensitizing gene and ionizing radiation may be delivered in the same or different hollow seeds.

Furthermore, the invention provides a novel method for delivery of non-nucleic acid therapeutic agents to target sites, in particular therapeutic agents, e.g., biologically active proteins or polypeptides, such as cytokines, growth factors, immunotoxins, therapeutic antibodies, hormones, et seq., by administrating a small hollow seed, preferably made of metal or polymeric material, having encapsulated therein said therapeutic agent, and visually confirming precise placement of the device, e.g., by stereotaxy, ultrasound, CT or MRI guidance.

The present invention improved methods for treating prostate cancer and brain tumors comprising the in vivo delivery of small hollow seeds, preferably made of metal or polymeric material, having encapsulated therein therapeutic nucleic acid sequences, in particular radiation sensitizing genes, optionally in conjunction with ionizing radiation. In particular, these methods will be used to treat subjects having cancer reoccurrence after radiation or drug therapy.

### BRIEF DESCRIPTION OF THE INVENTION

The accompanying drawings, which are incorporated in and form a part of the specification, illustrate embodiment of the present invention and, together with the description, serve to explain the principles of the invention. However, biocompatible polymers may be used also to produce such seeds.

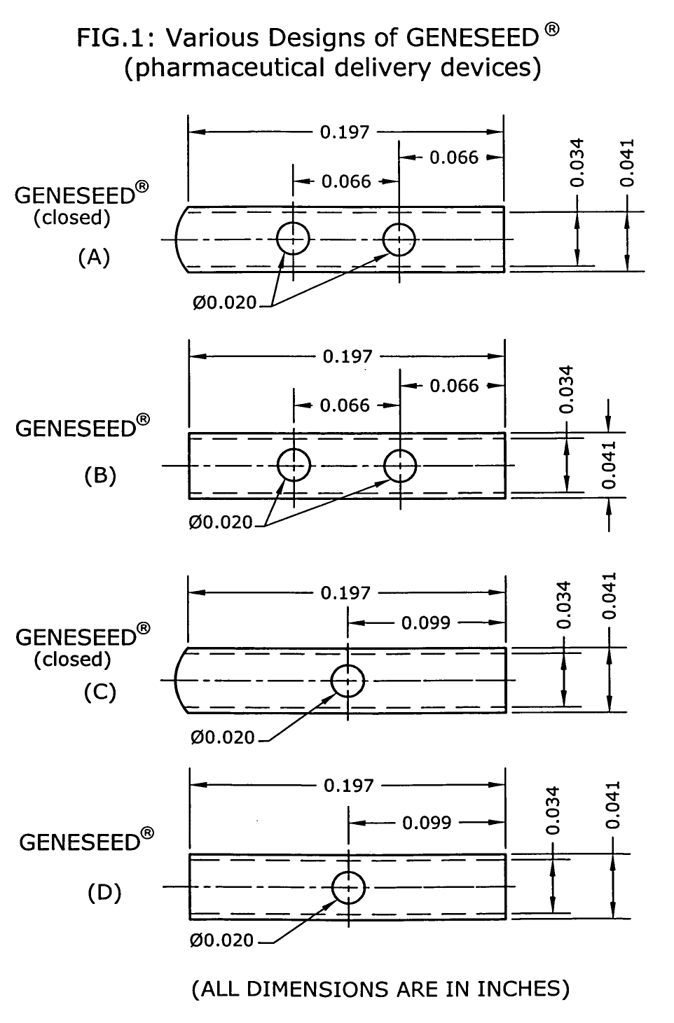

FIGS. 1A-1D are top plan views illustrating various designs of delivery devices according to the invention which are in the form of a tube open at one or both ends, and having one or two holes that allow for diffusion of encapsulated therapeutic agent, e.g., virus.

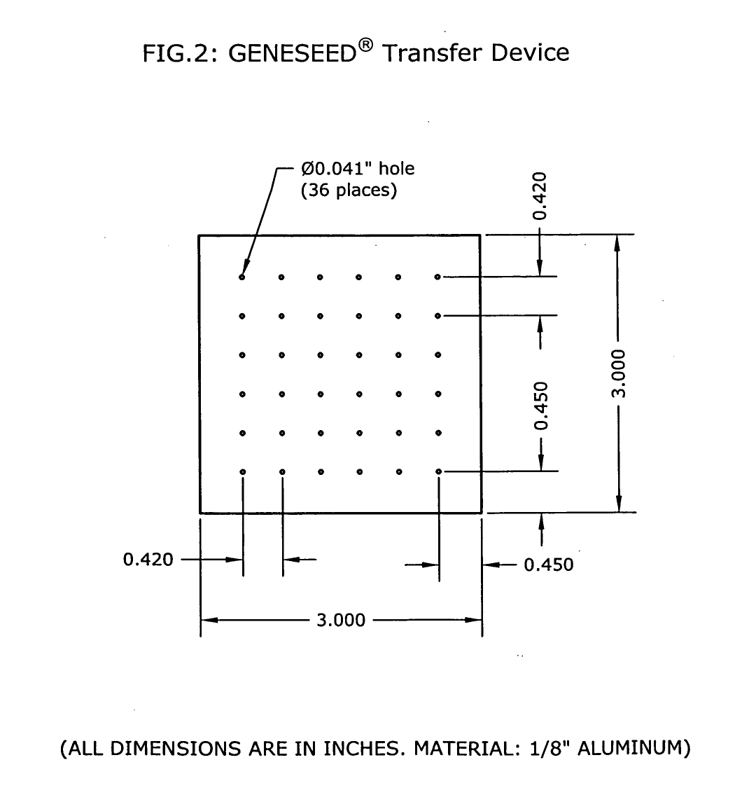

FIG. 2 is a top plan view illustrating a block containing small holes for storage of drug delivery devices according to the invention.

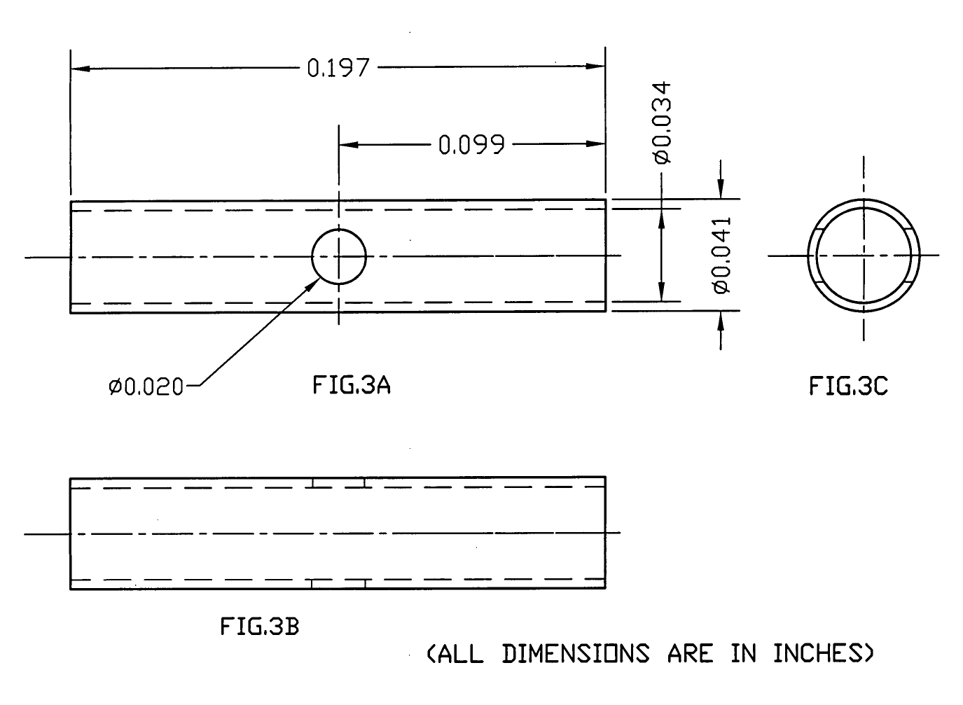

FIGS. 3A-3C are top plan, side and sectional views, respectively, illustrating a tube for use in the invention having a length of 0.197 inch, wall thickness of 0.0035 inch, diameter of 0.041 inch, and round hole having a diameter of 0.020 inch.

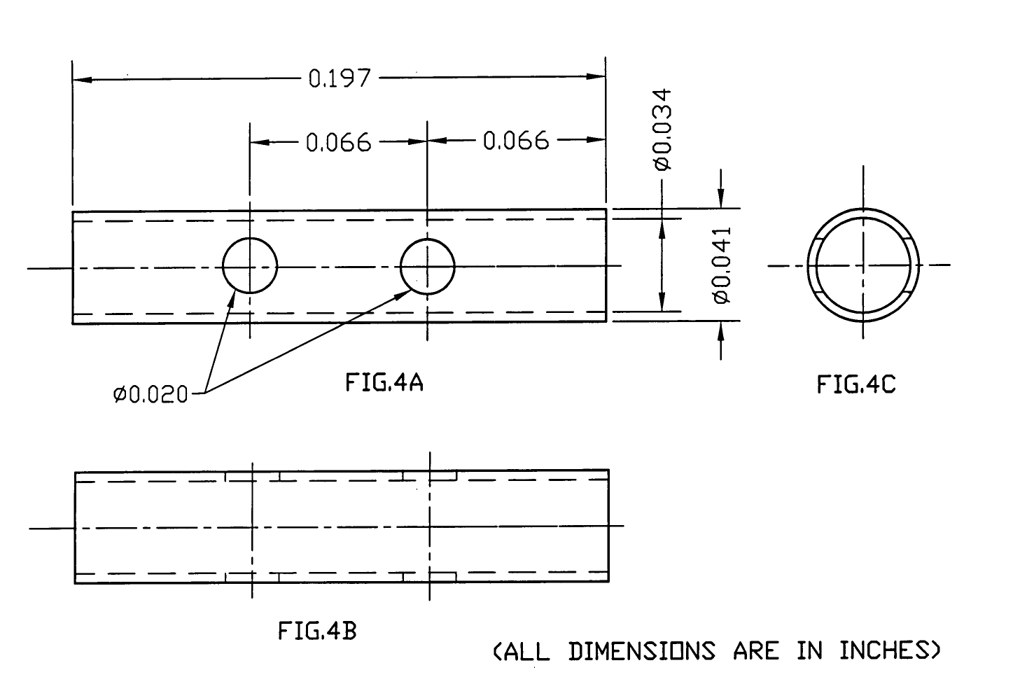

FIGS. 4A-4C are top plan, side and sectional views, respectively, illustrating another tube design having a length of 0.197 inch, a wall thickness of 0.0035 inch, a diameter of 0.41 inch, and two round holes having a diameter of 0.020 inch.

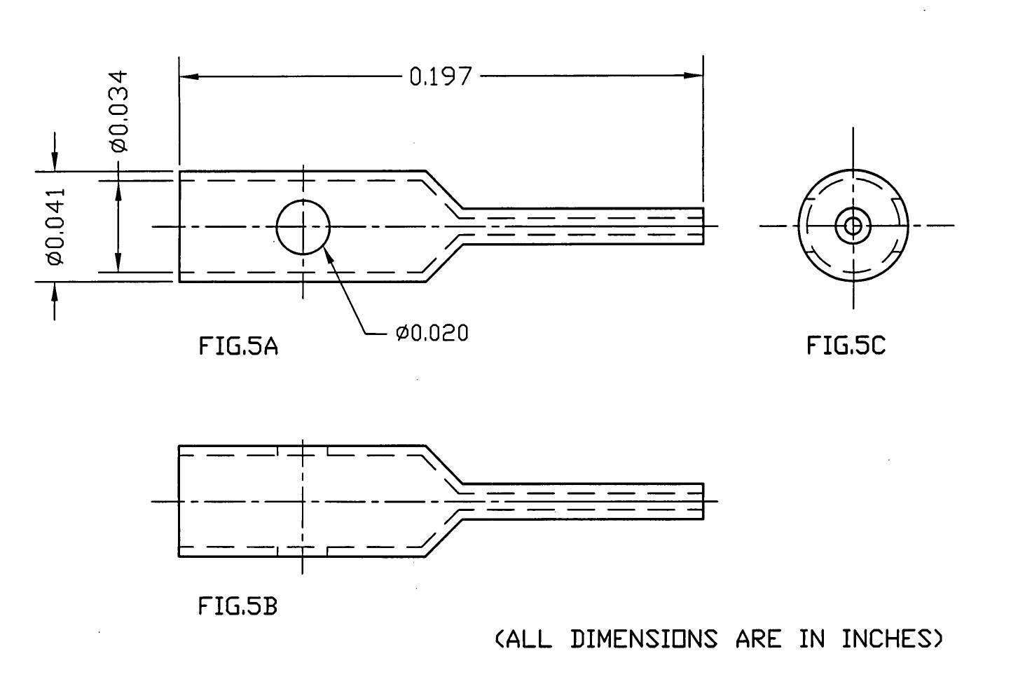

FIGS. 5A-5C are top plan, side and sectional views, respectively, illustrating a different tubular bottle-like design having a length of 0.197 inch, which is of comprised of two sections of differing diameter, wherein the larger diameter portion (0.041 inch in diameter) comprises a hole (0.020 inch in diameter) allowing for diffusion of encapsulated active agent, and taper into a smaller diameter portion (diameter of 0.02 inch), and wherein the wall thickness of both portions is 0.0035 inch.

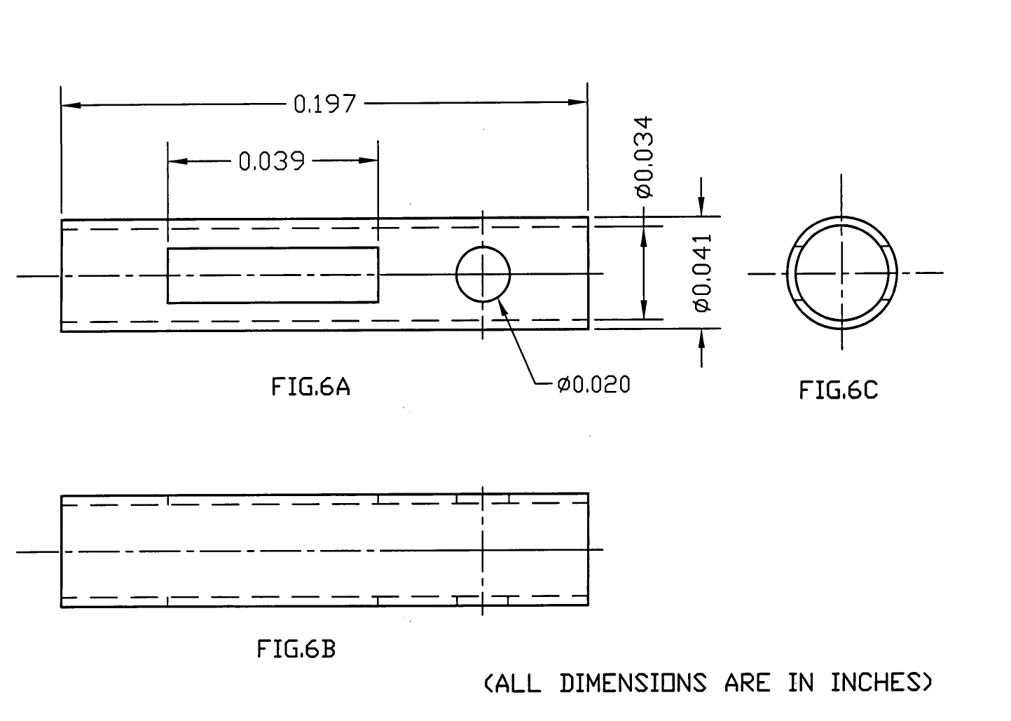

FIGS. 6A-6C are top plan, side and sectional views, respectively, illustrating another tubular design having a length of 0.197 inch, a wall thickness of 0.0035 inch, a hole allowing for diffusion which is 0.020 inch in diameter, and having a tube diameter of 0.041 inch.

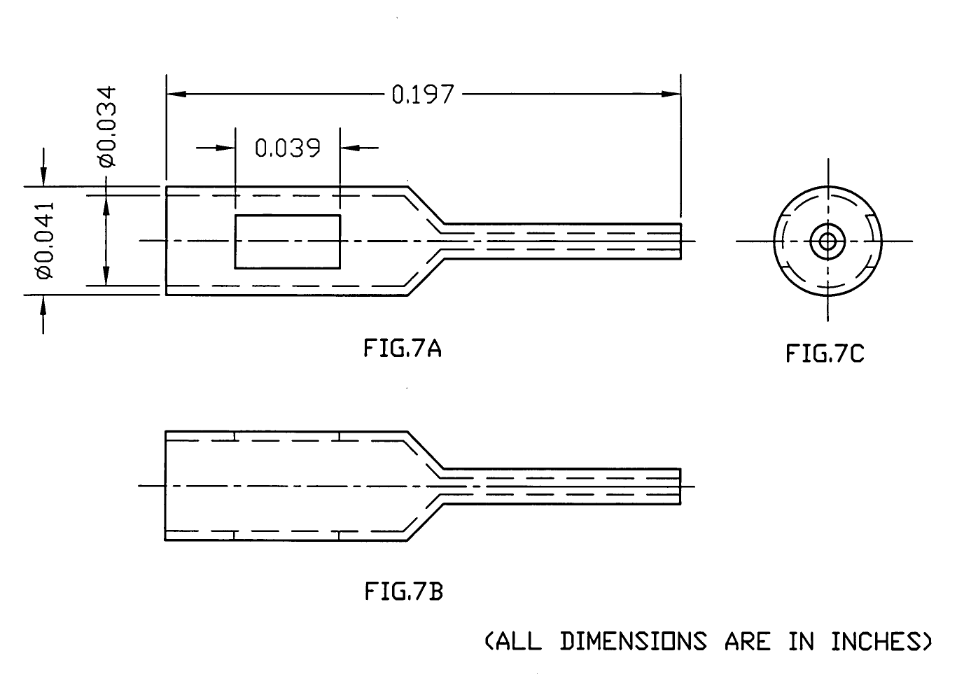

FIGS. 7A-7C are top plan, side and sectional views, respectively, illustrating another tubular design (bottle-like configuration) having an overall length of 0.197 inch, a wall thickness of 0.0035 inch, and a diameter of 0.041 inch (larger diameter portion), with a rectangular opening of 0.039 inches in length.

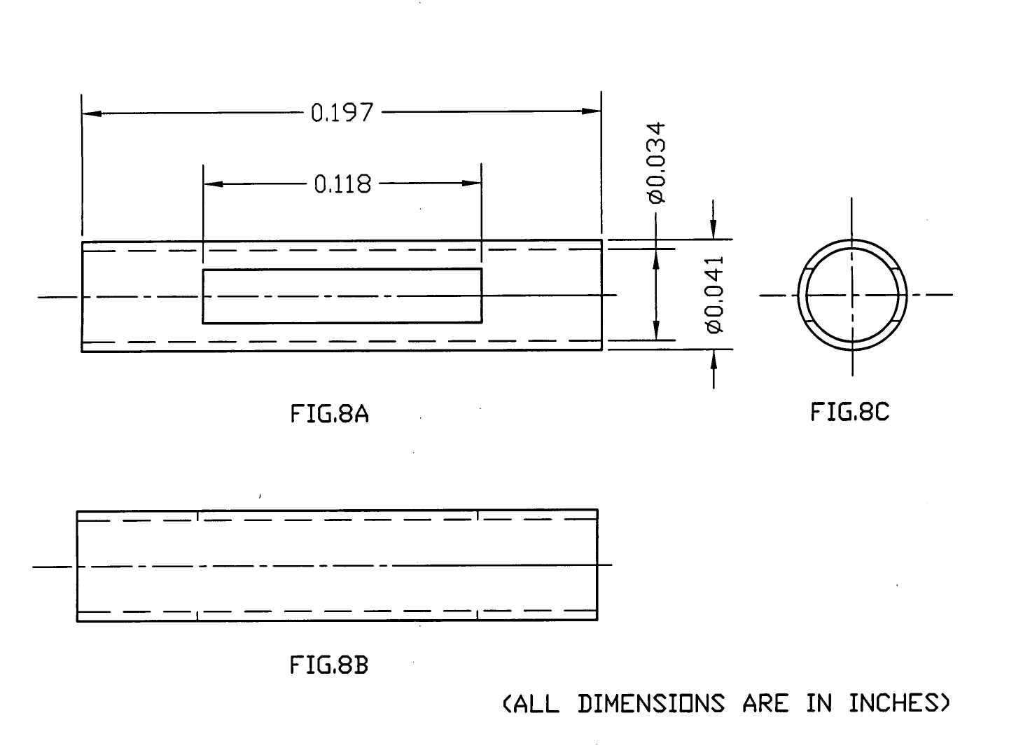

FIGS. 8A-8C are top plan, side and sectional views, respectively, illustrating another tube design having an overall length of 0.197 inch, a wall thickness of 0.0035 inch, a diameter of 0.041 inch, and a rectangular opening 0.197 inches in length.

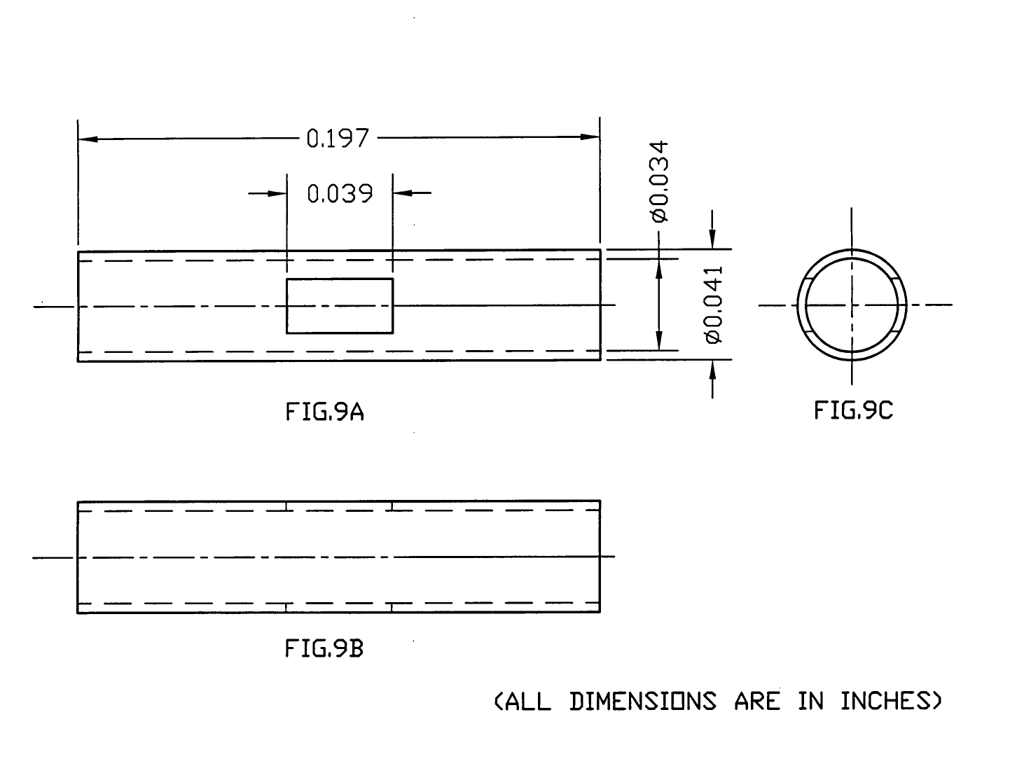

FIGS. 9A-9C are top plan, side and sectional views, respectively, illustrating yet another tube design having a length of 0.197 inch, wall thickness of 0.035 inch, diameter of 0.041 inch, and a rectangular opening 0.197 inches in length.

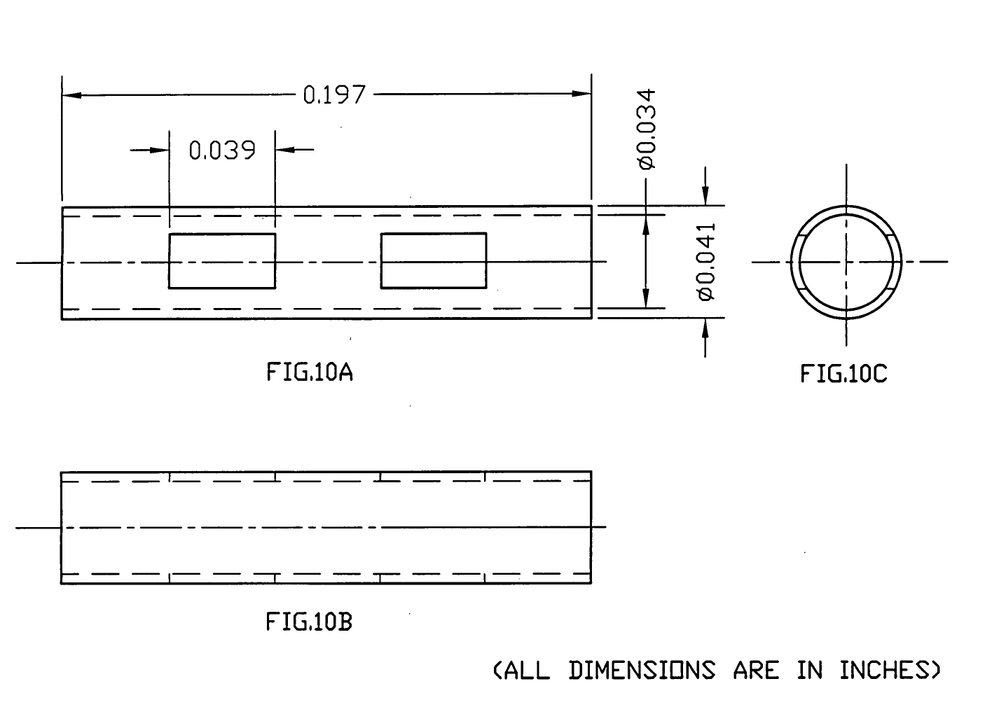

FIGS. 10A-10C are top plan, side and sectional views, respectively, illustrating another tubular design having a length of 0.197 inch, a diameter of 0.41 inch (overall), wall thickness of 0.035 inch, and two rectangular holes 0.039 inch in length.

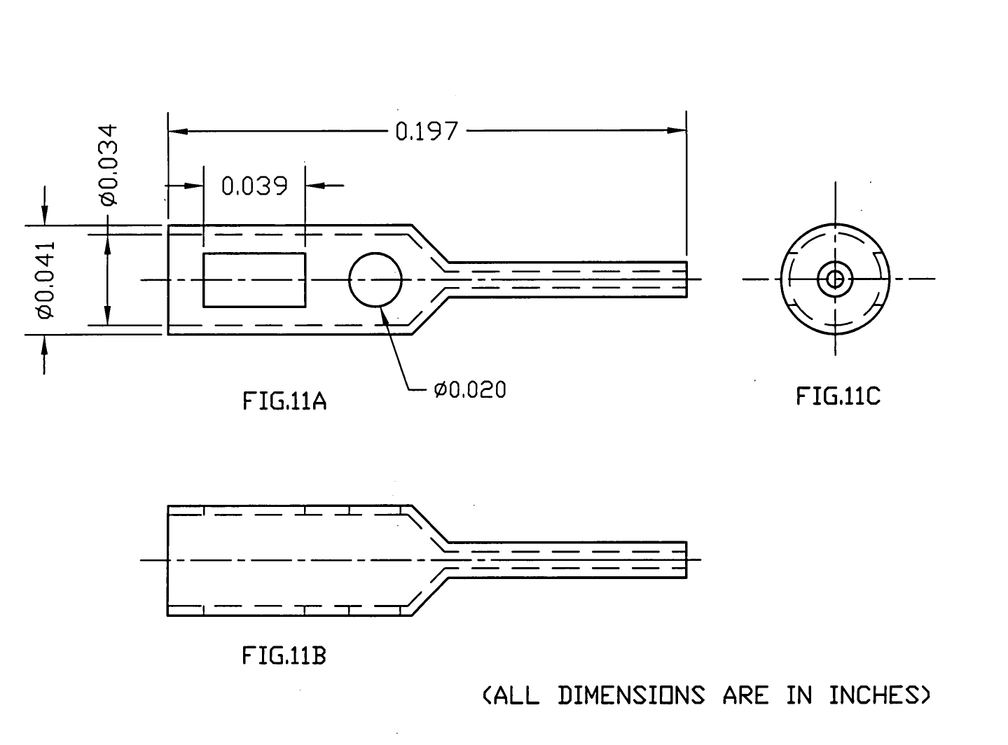

FIGS. 11A-11C are top plan, side and sectional views, respectively, illustrating another bottle-like tubular design having an overall length of 0.197 inch, diameter of 0.041 inch (large portion), wall thickness of 0.035 inch, rectangular opening that is 0.039 inch long and a circular opening 0.020 inch in diameter.

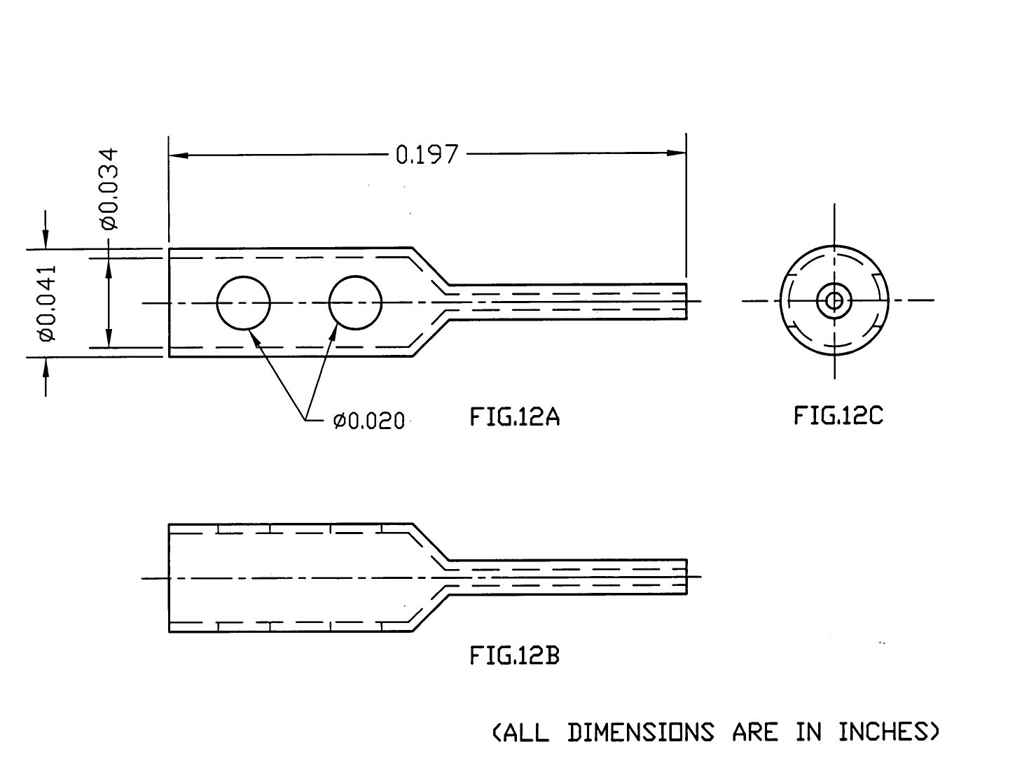

FIGS. 12A-12C are top plan, side and sectional views, respectively, illustrating another bottle-like tubular design having an overall length of 0.197 inch, a diameter of 0.041 inch, wall thickness of 0.035 inch, and two round holes that are 0.020 inch in diameter.

### DETAILED DESCRIPTION OF THE INVENTION

The present invention provides novel delivery systems for targeting nucleic acid sequences and other therapeutic agents to a target site, e.g., a tumor, that essentially comprises small hollow containers (or seeds), preferably constituted of a metal, metal alloy, or biocompatible polymer, e.g., biodegradable polymer, having encapsulated therein at least one nucleic acid sequence, or another therapeutic agent, e.g., a biologically active protein or polypeptide, such as a cytokine, hormone, growth factor, immunotoxin, cytotoxin, antibody, therapeutic enzyme or combinations/conjugates thereof, et. seq. According to the present invention, the small hollow seed, e.g., made of metal, is delivered to a precise site in a tissue, e.g., a tumor, by interstitial delivery methods such as implantation gun, syringe, or catheter, which methods further include visual confirmation, e.g., by stereotaxy, ultrasound, CT or MRI guidance, to ensure precise (millimeter precision) placement of seeds. These seeds, preferably made of metal or polymeric material, will be of a hollow configuration having one or more holes disposed therein that enable the hollow seed to be effectively delivered to desired sites, wherein they release a therapeutic agent (e.g., nucleic acid sequence) by diffusion.

An embodiment of the present invention is a small tube, preferably of metal or polymeric material, that may be open at one or both ends, having a length varying from 0.02 to 2.0 inch, more preferably from 0.05 to 0.5 inch, and most preferably from 0.16 to 0.25 inch; a diameter ranging from 0.004 to 0.2 inch, more preferably ranging from 0.01 to 0.10 inch, and most preferably ranging from 0.015 to 0.050 inch; a thickness ranging from 0.0005 to 0.5 inch, more preferably from 0.001 to 0.2 inch, and most preferably from 0.002 to 0.008 inch; and having one or more holes, e.g., round or rectangular, that allow for diffusion of the therapeutic agent from the tube, e.g., ranging from 0.006 to 0.18 inch in diameter, more preferably from 0.015 to 0.025 inch in diameter, and most preferably ranging from 0.01 to 0.03 inch in diameter.

Another embodiment of the subject hollow seed delivery system is a hollow metallic tube having a length of 0.197 inch, diameter of 0.041 inch, wall thickness of 0.0035 inch, and comprising one or two holes having a diameter of about 0.020 inch.

However, it is anticipated that other hollow seed configurations may be suitable for use in the invention. Examples thereof include rectangular, spherical, square, oblong, and combinations thereof. The most important aspects of the subject hollow seed delivery system are that it must be of a size that allows for precise interstitial delivery, e.g., as confirmed by stereotaxy, ultrasound, CT and MRI, and further should have one or more openings that allow for controlled diffusion of an encapsulated therapeutic agent there from, e.g., viral DNA. These openings can also be of various configurations, including rectangular, square, spherical, oblong, and combinations thereof. The only critical feature is that such openings must be of a size and configuration, which allows for diffusion of the encapsulated therapeutic agent, e.g., a nucleic acid at the desired diffusion rate for effective therapy.

The seeds will preferably be constituted of a metal or metal alloy that is suitable for in vivo usage, and which further exhibits the desired mechanical characteristics, i.e., may be formulated into desired configuration and interstitially delivered to a target site such as a tumor. Examples of such metals and metal alloys include those comprising platinum, titanium, stainless steel, silver, gold, and other known biocompatible and/or tissue absorbable metallic materials. Preferred metals, because of cost, biological, and mechanical properties, for construction of the metal seed, are stainless steel and high purity titanium metals.

More preferably, the titanium grade metal will be specified in the American Society for Testing of Materials F67-69, “Standard Specifications for Unalloyed Ti for Surgical Implications.” Titanium of such grade has been used for surgical implants for interstitial treatment of cancer. Registry numbers of suitable titanium materials include NR-460-S-165-S; NR-460-S-160-S; and GA-645-S-101-S.

As noted, the hollow seeds can also be constituted of polymeric materials, preferably biodegradable polymeric materials. Suitable polymers are well known to those skilled in the art and include, by way of example, polypropylene, polybutylene, polyvinylpyrrolidine, etc. The synthesis of such polymers and construction in desired hollow seed configurations according to the invention is well within the skill of the ordinary artisan.

Seeds with different types of perforations allow drugs to be released at different rates, e.g., rectangular holes can be used to release chemotherapeutic drugs to be released intratumorally at a fast rate. Spherical/circular holes can be used to deliver biologics at a relatively slow rate at the tumor site. The subject seeds, which are alternatively referred to as “GENESEED® pharmaceutical delivery devices”, can also be filled with a cocktail of drugs containing genetic drugs (viruses, plasmids, etc.), chemotherapeutic drugs, radionuclides, toxins, cytokines, therapeutic enzymes, antibiotics, antibodies, and conjugates/combination thereof, etc. The tubes preferably will be made of stainless steel, gold, titanium, platinum, or other biocompatible metals or an alloy of metals. The tubes can also be made of a suitable biocompatible polymeric material.

A further desirable characteristic of the subject hollow seed delivery system is that it can be frozen to very low temperatures, i.e., about −70° C., after a desired therapeutic agent, e.g., virus-containing solution, has been placed in the tube without affecting the desired properties of the hollow seed. Accordingly, the subject seeds may be kept frozen until they are to be introduced into patients, thereby maintaining stability and minimizing the risk of biocontamination. The seeds containing the therapeutic agent may itself be kept in frozen state, or it may be placed in specifically fabricated metallic cartridges that me be kept at very low temperatures. Seed cartridges suitable for storage of radioactive seeds are commercially available in the brachytherapy industry (Best Industries, Inc., Springfield, Va.; Micks Radio Nuclear, Bronx, N.Y.; Manan Medical, Northbrook, Ill., etc.), and may be modified, e.g., as need be, so that they may be kept at very low temperatures.

For example, titanium seeds according to the invention can be placed in transfer devices which comprise rectangular aluminum blocks suitable for freezing at −70° C. that contain holes suitable for insertion of titanium metal seeds.

The manufacturing of the hollow seeds used in the invention may be effected by known methods. One manufacturing having particular expertise in such manufacturing is BEST Industries, Inc., in Virginia, which has been manufacturing and distributing medical devices and radioisotopes since 1977. In particular, the company has extensive experience in the manufacture of radioactive seeds for implantation into cancer patients. However, one of ordinary skill in the relevant art can utilize known methods and materials to construct metal seed devices for use in the invention. Preferably, after manufacturing, the seed will be washed, autoclaved and dried prior to insertion of the desired therapeutic agent.

The hollow seed will then be encapsulated with the desired therapeutic agent, e.g., nucleic acid sequence or therapeutic protein or polypeptide, such as a cytokine or other cytotoxic materials such as chemotherapeutic drugs, toxins, therapeutic enzymes, conjugates, or radiolabeled materials. This may be effected, e.g., by insertion of a syringe needle of suitable diameter containing therapeutic agent (14G-26G needles) into the device. This may be effected by an automatic dispersing device. Preferably, the metal seed containing the material will then be frozen, e.g., at −70° C., until in vivo usage to maintain sterility and stability.

Before freezing the filled tube, it may be coated in order to ensure encapsulation of the therapeutic agent until delivery of the seed to the desired site in the affected body. The coating may be such that it will thermally degrade upon entering the body. Such coatings may be selected from polymers such as polydextrans, polyvinylpyrrolidone, poly(bis(p-carboxyphenoxy)-propane) and copolymers derived thereof, and biopolymers such as gelatin, human serum, albumin, cellulose, etc. Alternatively, the coating may decompose upon irradiation. For examples of such coatings, See U.S. Pat. No. 4,674,480, incorporated herein by reference. U.S. Pat. No. 4,674,480 also describes the use of antibodies on the seed surface to target the seed to targeted antigen-expressing cells in the affected body. The coating may also include a means of identifying or tracking seeds, such as a radioactive label as known to one of ordinary skill in the art.

The therapeutic device or seed may be delivered by any method known to one of most ordinary skill in the art. For example, the tube may be implanted or inserted by use of an implantation gun, catheter, syringe or the like. It is preferable that the delivery of the seed include visual confirmation of its placement by such means as stereotaxy, ultrasound, CT or MRI. Preferentially, the seeds are spaced closely together, such as at a distance of 3 to 5 mm between seeds in a uniform distribution pattern. Other distribution patterns may be selected depending on the area specific ailment being treated, as known to one of ordinary skill in the art.

After delivery of the seeds, the contents of the seeds diffuse from the seed to the surrounding tissue in the affected site. If a coating was placed on the seeds, diffusion will occur after thermal or nuclear degradation of the coating.

The seed described here may be filled with a therapeutic substance in order to treat various conditions, in particular cancer. The treatment of cancer may be effected by causing radiation sensitization of the affected tissue, and/or by genetic therapy of the affected area. One of ordinary skill in the art will understand that the therapeutic dosage will depend upon the therapeutic agent chosen, the size and site of the cancerous tumor being treated, and the relative age, weight and health of the patient. Usually the effective dose is delivered in an amount of approximately 0.1 ml in volume. The concentration of the therapeutic agent must therefore be adjusted in order to release an effective amount within the volume defined by the seed. A typical effective dosage will range from about 0.00001 gram to 10 grams of the active agent, e.g., a therapeutic nucleic acid sequence, protein, or polypeptide.

In the preferred embodiment, a metal seed will comprise a therapeutic nucleic acid sequence, e.g., a radiation sensitizing gene, antisense DNA, ribozyme, virus, plasmid, et seq. In an especially preferred embodiment, the seed will be used to deliver a combination of a radiation sensitizing gene, and ionizing radiation. Examples of radiation sensitizing genes are known in the art.

Suitable viral vectors that may be contained in the subject seeds include retroviral vectors, adenoviral vectors, and herpes simplex vectors.

Nucleic acid sequences that may be contained in the subject seeds include, by way of example, those that encode angiogenesis inhibitors, cytokines, apoptosis inducers, cell growth inhibitors, genes that affect cell cycle, toxins, hormones, enzymes, et seq.

Examples of other therapeutic agents (non-nucleic acids) that may be incorporated into the subject seed delivery device include cytokines such as TNFá, TNFâ, interleukins, interferons such as alpha, beta, gamma, colony stimulating factors, cytotoxins, hormones, cell growth inhibitors, therapeutic enzymes, et seq.

In a preferred embodiment, the subject seed delivery system will be used to treat cancers including, e.g., those of the central nervous system, prostate, head and neck, liver, pancreas, breast, uterine, lung, bladder, stomach, esophagus, and the colon.

However, the present invention should also be suitable for treatment of other conditions, e.g., by inflammatory conditions by targeting sites of inflammation with anti-inflammatory agents, infection by targeting sites of infection with anti-infectious agents such as antibiotic, antiviral, antifungal, etc. For instance, the subject seed delivery system can be interstitially delivered to the lung to deliver high dosages of antibiotics with persons suffering from pneumoniae.

As noted, an especially preferred usage of the invention is for treatment of cancer subjects who have relapsed after radiation therapy. These subjects are preferably treated with a seed containing a radiation sensitizing gene, and ionizing radiation. The radiation source may be a radionuclide such as iridium-192, iodine-125, palladium-103, yttrium-90, cerium-131, cerium-134, cerium-137, silver-111, uranium-235, gold-148, phosphorus-32, carbon-14, and other isotopes of rubidium, calcium, bismuth, barium, scandium, titanium, chromium, manganese, iron, cobalt, nickel, copper, zinc, zirconium, indium, yttrium, cadmium, indium, the non-earths, mercury, lead, americum, actinium, and neptunium. The dosage of radioactivity will be sufficient to elicit a therapeutic effect, e.g., anti-tumor effect. T he dosage will vary dependent upon the particular radioisotope, and other factors such as weight, disease, and overall condition of the patient treated.

The efficacy of the subject hollow seed delivery system for delivering a therapeutic moiety, e.g., nucleic acid sequence, will be confirmed in xenograft animal models. For example, mice will be implanted with human tumors, such as breast cancer, and squamous carcinoma, and then treated with seeds according to the invention that comprise a nucleic acid sequence or a cytokine and a source of ionizing radiation.

The above-described novel therapeutic device will now be described in the following example. It should be understood that the invention is not limited to the specific embodiments described above or to the example as set forth below, but is defined by the following claims in light of the description herein.

### EXAMPLE

A. Seed Design

The first step in this process is to optimize seed design to satisfy identified clinical needs. Although we have made some prototype seeds, variables include seed size, shape, and number of holes to provide portals for diffusion. Batches of 200 seeds will be manufactured for described experiments in an animal tumor model.

The prototype GENESEED® pharmaceutical delivery device consists of a metallic tube made of high purity titanium metal suitable for medical applications with a thickness of 0.005 inch. Low weight, high strength titanium is the metal of choice for the majority of implantable devices. Titanium grade metal specified in the American Society for Testing of Materials F67-69 “Standard Specifications for Unalloyed Ti for Surgical Implant Applications' will be used. Titanium of the same grade has been in use in surgical implants for interstitial treatment of cancer. Please refer to registry of sealed sources and device document number: NR-460-S-165-S, NR-460-S-160-S and GA-645-S101-S. The tube will be either closed on one end or both ends may be open. The titanium tube will contain one or two holes of diameter 0.5 mm (see FIG. 1). We will investigate the different designs in order to determine the optimum seed configuration for gene delivery. The different designs that we have considered include the following:

a. titanium tube with one end open, with two holes of diameter 0.5 mm

b. titanium tube with both ends open, with two holes of diameter 0.5 mm,

c. titanium tube with one end open, with one hole of diameter 0.5 mm

d. titanium tube with both ends open, with one hole of diameter 0.5 mm

We have selected the following design for these initial studies:

The titanium tubes of length 5 mm will be used, and the diameter will be 1.0 and 2.0 mm. Volumes of approximately 1 to 4 ì l of the viral solution can be easily placed in the seeds. Volume of genetic material placed in the seed can be varied by modifying the length or diameter of the tube.

The sterilized seeds will be suitable for freezing at the time of viral loading for ease of storage and to maintain viral viability, Nyberg-Hoffrman C, Aguilar-Cordova E. Instability of adenoviral vectors during transport and its implication for clinical studies, Nature Med 5:955-957, 1999. Since viruses are generally stored frozen (−70° C.), the suitability for GENESEED® pharmaceutical delivery devices to act as preloaded storage vessels suitable for use as needed is an added benefit. The titanium seeds containing the viral material will be placed in special transfer devices. These transfer devices are aluminum blocks of rectangular shape suitable for freezing at −70° C. These blocks contain small holes for the storage of GENESEED® pharmaceutical delivery devices (FIG. 2).

GENESEED® pharmaceutical delivery devices will function as delivery devices to freeze the biological material and transfer it to the hospital in the frozen state until ready for use in patients. If needed, the delivery devices can be placed in specially fabricated metallic cartridges and kept at very low temperatures. Seed cartridges for storage of radioactive seeds are already available in the brachytherapy industry and these cartridges can be modified for low temperature applications.

B. Seed Manufacture

High purity titanium tubes (medical grade metal) are cut to required size (±3%). The seeds will then be washed with an aqueous solution containing a mild detergent followed by acetone and sterile water for injection. The washed seeds will be dried in an oven at 110° C. for about two hours. Autoclaving will be performed to assure sterility. The seeds will be allowed to cool to room temperature. The viral solution will be added to the seed, using specially designed transfer devices, which are adaptable to robotic control. The transfer device containing GENESEED® pharmaceutical delivery devices will be kept frozen at −70° C. until ready for use in animals. Small numbers of seeds can be prepared manually for initial preclinical studies. Once a suitable configuration is identified, large scale manufacturing of GENESEED® pharmaceutical devices can be performed employing the proprietary technology developed by Best Industries Inc. and is currently in use for the production of iodine and palladium brachytherapy seeds, Suthanthiran K., Device and method for encapsulating radioactive materials, U.S. Pat. No. 4,891,165, Jan. 2, 1990. This method employs an automated dispensing device to add drug to seeds. It is of particular interest that much of the currently available radioactive seed implant technology will be directly adaptable for use with “GENESEED”.

C. Gene Vectors

The introduction of DNA vectors capable of expression in human cells forms the basis underlying gene therapy. The complexity of vectors that are capable of carrying DNA into cells ranges from plasmids, independent self-replicating circular DNA molecules, through adeno and herpes viruses. Typically, gene engineering is used to modify the viral genes to make viruses incapable of replication.

The recent development of conditionally-replicating oncolytic vectors for cancer therapy has introduced a new avenue of treatment for cancers that have been relatively refractory to standard forms of therapy, Kenney S, Pagano J, S, Viruses as oncolytic agents: a new age for “therapeutic” viruses? J. Nat Cancer Inst. 86: 1185-1186, 1994. Moreover, whereas both replication-defective vectors and chemotherapeutic drugs have their highest tumor tissue levels soon after injection and then decline at a rate dependent upon the particular agent, conditionally replicating oncolytic vectors which confine replication to the cancer tissue can multiply over time and spread throughout the tumor in order to achieve an improved therapeutic effect. Various strategies have evolved to design such vectors in a way that is effective in killing the cancer but does not cause harm to the normal tissues, Martuza R. L, Malick A., Markert J. M., Ruffner K. L., Coen D. M., 1991, Experimental therapy of human glioma by means of a genetically engineered virus mutant, Science, 252:854-856, 1991; Markert J. M., Coen D. M., Malick A., Mineta T., Martuza R. L., Expanded spectrum of viral therapy in the treatment of nervous system tumors, J. Neurosurg. 77:590-594, 1992. Herpes simplex had multiple advantages as a vector, including:

1) the ability to infect a wide variety of cell types from different species

2) a variety of animal models are available to test for efficacy and safety

3) antiviral drugs are available

4) the large size (153 Kb) can support large and multiple DNA inserts

5) high titers of virus can be generated

Dr. Robert Martuza had developed a vector, G207, which is a multiple-mutated conditionally-replicating herpes simplex virus-1 with deletions of both copies of 34.5 genes and a lacZ insertion disabling the gene for ICP6, Chou J., Kern E. R., Whitley R. J., Roziman B., Mapping of herpes simplex virus-1 neurovirulence to the g 34.5 gene, a gene nonessential for growth in culture, Science, 250:1262-1266, 1990; Goldstein D. J., Weller S. K., Herpes simplex virus 1-induced ribonucleotide reductase activity is dispensable for virus growth and DNA synthesis: isolation and characterization of an ICP6 1acZ insertion mutant, J. Virol., 62: 196-2051, 1988. G207 can grow within and kill cancer cells without toxicity to normal cells including normal neural cells. G207 was initially designed for treating malignant nervous system tumors. Efficacy was initially demonstrated in both malignant glioma and malignant meningioma models and safety has been demonstrated following inoculation of G207 into the brains of mice and of primates known to be highly sensitive to HSV-1, Mineta T., Rabkin S. D., Yazaki T., Hunter W. D., Martuza R. L., Attenuated multimutated herpes simplex virus-1 for the treatment of malignant gliomas, Nature Medicine, 1:938-9, 1995; Yazaki T., Manz H. J., Rabkin S. D., and Martuza R. L., Treatment of human malignant meningiomas by G207, a replication-competent multimutated herpes simplex virus-1, Cancer Research, 55:4752-4756, 1995; Hunter W. D., Martuza R. L., Feigenbaum F., Todo T., Mineta T., Yazaki T., Toda M., Newsome J. T., Platenberg R. C., Manz H. J., Rabkin S. D., Attenuated, replication-competent, herpes simplex virus type-I mutant G207: Safety evaluation of intracerebral injection in non-human primates, J. Virology, (in press) 1999.

However, the growth of G207 is not restricted to nervous system cancers. It has been shown that G207 will grow well in human breast cancer, squamous cell head and neck cancer, and in human prostate cancer cells and that it is effective following intraneoplastic delivery in several animal models. Moreover, G207 is effective both in hormone-sensitive and in hormone-resistant prostate cancers and in tumors that have had or have not had prior radiotherapy. Because G207 can replicate in tumor cells and spread from cell to cell, better tumor distribution is possible than with replication-defective vectors. The efficacy of intraneoplastic administration of G207 for prostate cancer has been demonstrated and, in studies currently being concluded, intraporstatic inoculation of G207 has been safe in two standard animal models used for HSV toxicity testing: mice (Balb/c) and non-human primates (aotus). Conditionally-replicating herpes viruses are novel vectors ideally suited for this innovative form of prostate cancer therapy. A Phase I study of G207 is now being completed which demonstrates that this conditionally-replicating herpes vector can be inoculated directly into the human brain at titers as high as 3×10⁹ pfu without neural or systemic toxicity. A phase II trial of G207 for malignant gliomas is now being planned. We anticipate that within this next year an IND for human trials of intraprostatic inoculation of G207 to treat post-radiation local recurrences will be filed. The studies designed herein may extend this concept to allow more accurate delivery of the vector within prostatic, brain, or other tumors and tissues.

D. Experiments

1. Optimization of the Design of GENESEED® for Interstitial Delivery of Viral Vectors and Cytokines

The four different types of GENESEED® pharmaceutical delivery devices described in FIG. 1 will be filled with viral solutions and frozen at −70° C. The seeds will be implanted interstitially in mice bearing tumor xenografts (prostate tumor models). Melting and release of viral solution occurs rapidly. At selected time points post implantation, the animals will be sacrificed and the tumor will be excised. The extent of diffusion and virus entry into tumor cells will be evaluated using histochemistry. The optimum design will slowly diff-use the viral material, allowing maximal intracellular viral uptake in tumor cells.

Experiments to be performed Using Different Kinds of Seeds:

| Seed Design | Drug | Tumor Model |
|---|---|---|
| A. Two holes/One end open | 1. Virus | 1. Prostate Tumor |
| B. Two holes/Both ends open | | |
| C. One hole/One end open | | |
| D. One hole/Both ends open | | |

2. Human Prostate Tumor System

We will use human prostate cancer cell line-derived tumors from LnCaP in athymic mice to study the efficiency of the use of GENESEED® to deliver G207, a lacZ containing vector, versus direct inoculation of vector, a procedure with which we have prior experience. Three mice will be used for each time point. Tumors will be generated as noted in the methodology section. When tumors are 100 mm³ or larger in size, they will be inoculated either with a GENESEED® or with a standard inoculation needle containing either virus or buffer solution and using similar volumes and pfus of virus. The goal will be approximately 10⁶ to 2×10⁷ pfu but the actual amounts will be determined by the capacities of the GENESEED® used and the titers of the virus solutions. At days 1,2,3 and 7, after inoculation, animals will be sacrificed and tumor sections will be examined for the distribution of lacZ expression. Hematoxylin and eosin staining will also be performed to determine areas of necrosis and to view cellular morphology. Preliminary experiments have shown that the virus does not become systemic following interstitial injection, however, animal organs including lungs, liver, and brain will also be sectioned and scored.

### Experiment 1

Specific Aim I will be addressed with the following experiment.

Evaluation of viral distribution within tumors as a
function of time after GENESEED ® pharmaceutical
delivery devices implant
Time

| | | | | | | 2 | 3 | 7 |
|---|---|---|---|---|---|---|---|---|
| | | 0 | 4 h | 12 h | 24 h | days | days | days |
| Controls (buffer only, | | X | | | X | | | |
| all designs) | | | | | | | | |
| Controls, | | X | X | X | X | X | X | |
| intratumoral | | | | | | | | |
| injection | | | | | | | | |
| GENESEED ®, | A | X | X | X | X | X | X | |
| Design | B | X | X | X | X | X | X | |
| | C | X | X | X | X | X | X | |
| | D | X | X | X | X | X | X | |

Three mice will be used per time point. Controls and design A seed experiments will be performed for all time points in the initial experiment. Based on resultant data, designs B, C, and D will be studied at the most relevant time points after implantation. This strategy should reduce the necessary total number of mice. Similarly, controls will also be performed with designs B, C, and D seeds at selected time points.

Anticipated Results

Non-replicating vectors would be expected to be maximally distributed at early time points. Since G207 is a conditionally replicating vector, maximal distribution is anticipated at later time points. Our experimental plan will be modified accordingly once design A test samples and controls are examined. These experiments will only use 1 seed per tumor, with the expectation that multiple seed use in a tumor will similarly depend on optimal single seed design for viral release. The seeds will be loaded with 10⁶ pfus per seed. The controls include seeds with buffer only, as well as direct injection of Viral solution into the tumor. Comparisons of patterns of distribution will be made.

### Experiment 2

Specific Aim II will be addressed with the following experiment.

Tumor Growth Delay

The optimal seed design based on data from experiment #1 will be used in tumor growth delay studies

| | |
|---|---|
| 1. Controls #1 | Tumor bearing mice |
| 2. Controls #2 | PBS in seeds |
| 3. Controls #3 | Viral, direct intratumoral injection |
| 4. GENESEED ® | (optimal design) with virus |

Injections will be performed into −120-150 mm³ tumors as described. Eight mice will be used for each experimental group. Animals will be monitored for 30 days and tumor volume will 3 be plotted as a function of time. Animals will be sacrificed, on day 30 or when the tumor volume exceeds 1 cm³.

Anticipated Results/Interpretation of Data

We anticipate tumor growth delay to occur in GENESEED® and direct intra-tumor injected animals. If needed, additional experiments will be performed using more than one seed per tumor. The observation of tumor growth delay comparable to direct tumor injection will be the endpoint confirming the utility of GENESEED® pharmaceutical delivery device for viral vector delivery. Improved distribution experiments to show GENESEED® superiority over direct injection may require larger tumors in a large tumor model system and may be considered in a Phase II proposal.

Methodology

Cell Lines: LNCaP cells are maintained in IMEM containing 5% calf serum at 37° C. in 5% CO₂ with penicillin and streptomycin added to all media, and are tested to ensure freedom from mycoplasma contamination.

Subcutaneous Tumor Model: All animal procedures require approval by the Georgetown University Animal Care and Use Committee. The mice (6-to-7 week old male BALB/c/nu/nu for human tumors) are anesthetized with an i.p. injection of a 0.25-0.30 ml solution consisting of 84% bacteriostatic saline, 10% sodium pentobarbital (1 mg/ml: Abbott Laboratories, Chicago, Ill.) and 6% ethyl alcohol or inhalation of 2-3 minimal alveolar concentration of methoxyflurane. LNCaP tumors are induced by s.c. flank injection of 5×10⁶ LNCaP cells in 0.1 ml with an equal volume of Matrigel and LNCaP cells in suspension. Tumors are measured by external caliper to the 0.1 mm, and volumes are calculated (V=H×L×W). Once a tumor volume of approximately 120-150 mm 3 is reached, tumors are either inoculated with 5-10 ì l containing 10⁷ plaque forming units (pfu) G207 or virus buffer (150 mM NaCl, 20 mM Tris, pH 7.5). Experiments using seeds may require the placement of 1-2 GeneSeeds to deliver a comparable number of pfus. Controls will use GeneSeeds without virus. Tumor volumes are followed and recorded; animals are sacrificed when a tumor volume is greater than 1 cm³.

X-gal staining of tumors and tissues: The samples are snap frozen in isopentane cooled with dry ice. Cryostat sections of 10 um in thickness are prepared from each sample. Sections are fixed in 2% paraformaldehyde in PBS for 10 min, washed 3 times in PBS, and incubated with PBS containing 2 mM magenesium chloride, 0.01% sodium dexoycholate and 0.02% Nomidet P (NP)-40 at 4° C. for 10 min. Sections are further incubated with substrate solution (PBS containing 1 mg/ml X-gal, 5 mM potassium ferricynide, 5 mM potassium ferrocyanide, 2 mM magnesium chloride, 0.01% sodium dexoycholate and 0.02% NP-40) at 32° C. for 3 h, and then washed once with water and twice with PBS containing 2 mM EDTA. Sections are counterstained with hematoxylin and eosin before mounting.

Statistical Analysis

In vivo efficacy. The parameters measured during the study will conclude tumor volume and survival. Survival comparisons will be made to controls using the Kaplan-Meier method and Log Rank tests. Tumor size comparisons will be made to the control group using the F test.

E. Animal Models

All animal procedures are performed under a protocol approved by the IACUC of Georgetown University School of Medicine. This protocol has been submitted for review. Six-to-seven week old male BALB/C nu/nu mice will be used for human tumor (LNCap) xenografts. Detailed injection procedure is described under “subcutaneous tumor model” section of methodology.

Two hundred (200) animals are requested based on calculations for Experiment #1: 6 arms×7 time points×3 animals per point=126 animals and Experiment #2: 4 arms×8 animals per arm×2 experiments=64 animals. Animals are important for use with the xenograft model since we are dealing with interstitial tumor delivery system.

All animal injections will be performed with the sterile instruments and solutions. Animals will be anesthetized for procedures as described in the “subcutaneous tumor model” section.

Euthanasia will be performed using CO₂ asphyxiation according to the recommendations of the panel on euthanasia of the American Veterinary Medical Association. The reasons for its selection are: (a) the rapid depressant and anesthetic effects of CO₂ are well established; (b) it is inexpensive, noninflammatable, and nonexplosive, and presents minimal hazard to personnel when used with properly designed equipment; (e) it does not result in accumulation of tissue residues in food producing animals; (d) it does not distort cellular architecture.

The invention as exemplified herein will now be set forth in the following claims.

What is claimed is:

1. A drug delivery system, comprising:
A substantially hollow, polymeric container, said container being sized and adapted for insertion into a tissue or organ in vivo to within about 1 millimeter of a desired position within the tissue or organ, said container including
one or more openings therein, said openings being sized and arranged to provide for the controlled diffusion of a therapeutic agent out of said container, the therapeutic agent comprising a nucleic acid sequence, protein or polypeptide.

2. The drug delivery device of claim 1, wherein said container has a uniform cross-sectional area along a longitudinal axis.

3. The drug delivery system of claim 1, wherein said container has an elongated shape with varying width.

4. The drug delivery system of claim 1, wherein said container is substantially cylindrical in shape.

5. The drug delivery system of claim 4, wherein said openings are provided at respective ends of said cylindrical container.

6. The drug delivery system of claim 5, wherein said container has a length in the range of 0.002 to 3 inches, a diameter in the range of 0.004 to 4 inches, and a wall thickness in the range of 0.0005 to 0.5 inches.

7. The drug delivery system of claim 6, wherein said openings are round holes having an average diameter in the range of 0.002 to 0.2 inches.

8. The drug delivery system of claim 1, wherein said openings is round.

9. The drug delivery system of claim 1, wherein said openings have a shape selected from the group consisting of rectangular, square, spherical and oblong.

10. The drug delivery system of claim 1, said container further comprising a degradable coating provided on at least a portion of an exterior surface thereof, said coating being adapted to delay controlled diffusion of the therapeutic agent until said container is in the desired position within the tissue or organ.

11. The drug delivery system of claim 10, wherein said degradable coating is comprised of a polymer or combination of polymers selected from the group consisting of polydextran, polyvinylpyrrolidone, and poly(bis(p-carboxyphenoxy)-propane).

12. The drug delivery system of claim 10, wherein said degradable coating is comprised of a biopolymer.

13. The drug delivery system of claim 12, wherein said biopolymer is selected from group consisting of gelatin, human serum albumin, and cellulose.

14. The drug delivery system of claim 1, wherein the therapeutic agent further comprises a radionuclide.

15. A drug delivery system, comprising:
a substantially hollow container, said container being sized for insertion into a tissue or organ in vivo to within about 1 millimeter of a desired position within the tissue or organ, said container including one or more openings therein, said openings being sized and arranged to provide for the controlled diffusion of a therapeutic agent out of said container, the therapeutic agent comprising a nucleic acid sequence, protein, or polypeptide; and
a storage cartridge having one or more compartments, each of said one or more compartments being constructed and arranged to house one or more of said containers, said storage cartridge being capable of withstanding freezing to a temperature of about −70° C.

16. The drug delivery system of claim 15, wherein said storage cartridge comprises a metal block.

17. The drug delivery system of claim 16, wherein said storage cartridge comprises a rectangular metal block.

18. The drug delivery system of claim 16, wherein each of said one or more compartments is a hole through a portion of said metal block.

19. The drug delivery system of claim 15, wherein said container is formed of a metal.

20. The drug delivery system of claim 19, wherein the metal is titanium.

21. The drug delivery system of claim 15, wherein said container is formed of a biocompatible plastic.

22. The drug delivery device of claim 15, wherein the container has a uniform cross-sectional area along a longitudinal axis.

23. The drug delivery system of claim 15, wherein said container is substantially cylindrical in shape.

24. The drug delivery system of claim 23, wherein said openings are provided at respective ends of said cylindrical container.

25. The drug delivery system of claim 24, wherein said container has a length in the range of 0.0002 to 3 inches, a diameter in the range of 0.0004 to 4 inches, and a wall thickness in the range of 0.0005 to 0.5 inches.

26. The drug delivery system of claim 25, wherein said openings are round holes having an average diameter in the range of 0.002 to 0.2 inches.

27. The drug delivery system of claim 15, wherein said container has an elongate shape with a varying width.

28. The drug delivery system of claim 15, wherein said openings are round holes.

29. The drug delivery system of claim 15, wherein said openings have a shape selected from the group consisting of rectangular, square, spherical and oblong.

30. The drug delivery system of claim 15, said container further comprising a degradable coating provided on at least a portion of an exterior surface thereof, said coating being adapted to delay diffusion of the therapeutic agent until said container is in the desired position within the tissue or organ.

31. The drug delivery system of claim 30, wherein said degradable coating is comprised of a polymer or combination of polymers selected from the group consisting of polydexiran, polyvinylpyrrolidone, and poly(bis(p-carboxyphenoxy)-propane).

32. The drug delivery system of claim 30, wherein said degradable coating is comprised of biopolymer.

33. The drug delivery system of claim 32, wherein said biopolymer is selected from the group consisting of gelatin, human serum albumin, and cellulose.

34. The drug delivery system of claim 15, wherein the therapeutic agent further comprises a radionuclide.
